(19) United States
(12) Patent Application Publication
(10) Pub. No.: US 2019/0192109 A1
(43) Pub. Date: Jun. 27, 2019

(54) STETHOSCOPE

(71) Applicant: Joonhyeok CHOI, Seongnam-si (KR)

(72) Inventor: Joonhyeok CHOI, Seongnam-si (KR)

(21) Appl. No.: 14/759,858

(22) PCT Filed: Apr. 7, 2015
(86) PCT No.: PCT/KR2015/003466

§ 371 Date: Jul. 8, 2015

(51) Int. Cl. A61B 7/04

(52) U.S. Cl. CPC A61B 7/04

(57) ABSTRACT

A stethoscope according to exemplary embodiments of the inventive concept includes a chestpiece for auscultating a patient, a first headset unit enabling a doctor to listen to auscultation sound auscultated by the chestpiece, a second headset unit enabling a patient to listen to the auscultation sound, and a connection unit for connecting the chestpiece, the first headset unit, and the second headset unit with each other and guiding the auscultation sound to the first headset unit and the second headset unit. Such the structure enables for a patient to listen to the auscultation sound as well as for a doctor.

### TECHNICAL FIELD

Example embodiments of inventive concepts relate to a stethoscope, and more particularly, to a stethoscope enabling for a doctor and a patient to listen to the auscultation sound at the same time.

### BACKGROUND ART

Generally, a stethoscope is a medical device for auscultating inside of the human body and diagnosing illness of patient. It is used to listen to various inner body organs such as intestines and blood flow in arteries and veins as well as lung and heart sounds.

The stethoscope includes an auscultation member for auscultating inside of the human body, a headset for listening to the auscultation sound, and a guiding tube for guiding the auscultation sound by connecting the auscultation member and the headset. The stethoscope having such the structure is used to examine a patient through the auscultation sound received from the headset by contacting the auscultation member on the patient's body.

By the way, a doctor needs to provide his headset to the patient in the case that the patient is willing to listen to the auscultation sound in person. Accordingly, the doctor should endure inconvenience of sharing his headset with the patient.

### DETAILED DESCRIPTION OF THE INVENTION

### Technical Goal of the Invention

According to an exemplary embodiment, the inventive concept provides a stethoscope being capable of delivering the auscultation sound to a patient as well as to a doctor.

### Technical Solution of the Invention

According to an exemplary embodiment of the inventive concept, a stethoscope includes a chestpiece for auscultating a patient, a first headset unit for delivering the auscultation sound to a doctor's ears and a second headset unit for delivering the auscultation sound to a patient's ears, and a connection unit guiding the auscultation sound to the first headset unit and the second headset unit wherein the connection unit connects the chestpiece, the first headset unit, and the second headset unit mutually.

The connection unit is prepared among the chestpiece, the first headset unit, and the second headset unit and includes a first connecting tube and a second connecting tube which are connected to the chestpiece by one terminals and to each of the first headset unit and the second headset unit by the other terminals.

The connection unit is formed of a hollow tube of a “T” shape which is connected to the chestpiece, the first headset unit, and the second headset unit and enables to deliver the auscultation sound from the chestpiece to the first headset unit and the second headset unit at the same time.

The connecting tube further includes a Bluetooth connecting the first headset unit and the chestpiece in wireless.

According to another exemplary embodiment of the inventive concept, a stethoscope includes a chestpiece for auscultating a patient, a first headset unit and a second headset unit enabling to listen to the auscultation sound of the chestpiece, a connection unit prepared among the chestpiece, the first headset unit, and the second headset unit and guiding the auscultation sound from the chestpiece to the first headset unit and the second headset unit by branching the auscultation sound.

The connection unit includes a first connecting tube and a second connecting tube which connected to the chestpiece by one terminals and to each of the first headset unit and the second headset unit by the other terminals, wherein the one terminals of the first connecting tube and the second connecting tube are connected to the chestpiece across a connecting body.

According to yet another exemplary embodiment of the inventive concept, a stethoscope includes a chestpiece for auscultating a patient, a first headset unit connected to the chestpiece and delivering the auscultation sound from the chestpiece to a doctor's ears, a second headset unit connected to the chestpiece and delivering the auscultation sound from the chestpiece to a patient's ears, and a connection unit delivering the auscultation sound to the second headset unit by connecting the chestpiece and the second headset unit.

The connection unit is formed of a hollow tube shaped “T” each ends connected to the chestpiece, the first headset unit, and the second headset unit and branches the auscultation sound from the chestpiece for delivering the auscultation sound to the first headset unit and the second headset unit at the same time.

The connection unit includes a Bluetooth connecting the second headset unit and the chestpiece in wireless.

### Effect of the Invention

According to the inventive concept, a stethoscope enables for a patient to listen to the auscultation sound as well as for a doctor, thereby increasing reliability of diagnosing.

The stethoscope includes a connection unit of simple structure branching the auscultation sound, and accordingly, the auscultation sound may not leak before being delivered to the doctor's ears and the patient's ears directly.

### BRIEF DESCRIPTION OF THE DRAWINGS

FIG. 1 is a schematic perspective view of a stethoscope according to an exemplary embodiment of the inventive concept;

### BEST MODE FOR CARRYING OUT THE INVENTION

Various example embodiments will be described more fully hereinafter with reference to the accompanying drawings.

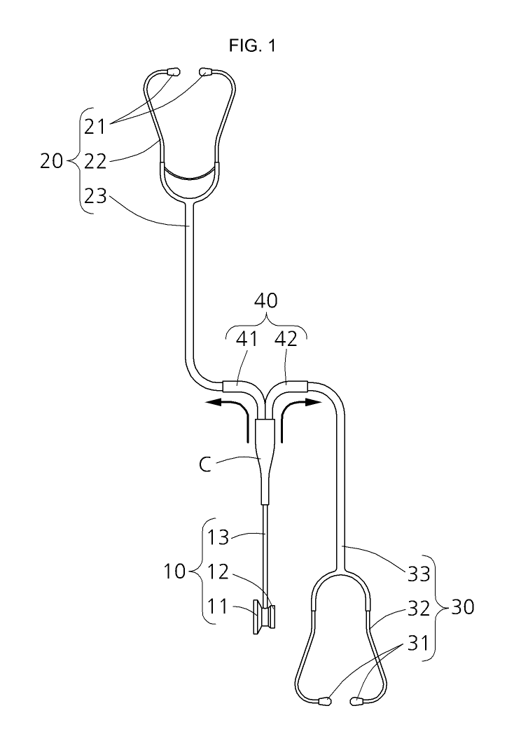

Referring to FIG. 1, a stethoscope 1 according to an exemplary embodiment of the inventive concept includes a chestpiece 10, a first headset unit 20, a second headset unit 30 and a connection unit 40.

The chestpiece 10 is used to examine the inside of human body while contacting the patient on the body. Specifically, the chestpiece 10 is used to examine the patient by listening to the internal sound of the body, for example, heart sound, breath sound, artery sound, intestine sound, and blood vessel sound. Also, the chestpiece 10 is contacted to the patient on the body to gather the brachial artery sound when measuring blood pressure. Such the chestpiece 10 includes a diaphragm 11 prepared at one side, a bell 12 prepared at the opposite side of the diaphragm 11, and an auscultation tube 13 for delivering the auscultation sound.

The diaphragm 11 includes a flat plastic vibrating plate and lets the auscultation sound of frequency 100 Hz to 1 KHz, for example, lung sound or intestine sound, be heard. The bell 12 has a bell shape and lets the auscultation sound of frequency 20 Hz to 200 Hz, for example, comparatively low sound such as opening and shutting sounds of valves of the heart and swirl and backward flow sounds of blood flow. That is, the diaphragm 11 and the bell 12 auscultate for sounds of each different frequencies to examine the patient exactly.

As a guide, the reason that the diaphragm 11 and the bell 12 auscultate for sounds of each different frequencies is due to the structure of sound collecting position. Specifically, the bell 12 may auscultate for sounds of low frequency because it has no vibrating plate so that the vibration sound generated from a body may vibrate air directly. On the other hand, the diaphragm 11 delivers vibration of skin through plastic vibrating plate so that the vibrating of the vibrating plate may vibrate air to deliver the sound. At this time, the sound of low frequency may not be delivered any more when being absorbed to the vibrating plate, therefore, the diaphragm 11 may auscultate for the sound of high frequency when the sound has more than a predetermined vibration.

The auscultation sound gathered by the diaphragm 11 and the bell 12 is delivered through the air, for that, the diaphragm 11 and the bell 12 are connected to auscultation tube 13. The auscultation tube 13 is a path for auscultation sound formed of flexible material.

The first headset unit 20 delivers the auscultation sound gathered from the chestpiece 10 to the doctor's ears. The first headset unit 20 includes a pair of first ear pieces 21 for the doctor's ears, a pair of first binaurals 22 extended from the first ear pieces 21, and a first headset tube 23 extended to be connected to the first binaurals 22.

The pair of ear pieces 21 are bent along the direction of external auditory meatus so as to be inserted and contacted to the doctor's ears. Such the first ear pieces 21 are preferred to be formed of material being flexible and harmless to humans to provide comfort even in the state of being inserted to the ears for a long time.

The first binaural 22 is a kind of tube connected from the first earpiece 21. Such the first binaural 22 delivers the auscultation sound transferred through the first headset tube 23 connected through the auscultation tube 13 and is made of metal such as reinforced aluminum, steel, or copper.

The first headset tube 23 is made of flexible material like the auscultation tube 13 described above and performs a function of pathway of the auscultation sound.

The second headset unit 30 enables for the patient to listen to the auscultation sound from the chestpiece 10 by being connected to the patient's ears. The second headset unit 30 includes a pair of second ear pieces 31, a pair of second binaurals 32, and a second headset tube 33 like the first headset unit 20. The second earpieces 31, the second binaurals 32, and the second headset tube 33 have similar structures with the first earpieces 21, the first binaurals 22, and the first headset tube 23 of the first headset unit 30, and detailed descriptions thereof will be omitted accordingly.

The connection unit 40 is located among the chestpiece 10, the first headset unit 20, and the second headset unit 30 and prepared to connect them with each other to guide the auscultation sound from the chestpiece 10 to the first headset unit 20 and the second headset unit 30. The connection unit 40 includes a first connecting tube 41 and the second connecting tube 42 which are separated to be connected to the auscultation tube 13 by one terminal of the each connecting tubes as shown in FIG. 1. and to the first headset tube 23 and the second headset tube 33 by the other terminal of the each connecting tubes. The first connecting tube 41 and the second connecting tube 42 have a shape of hallow hole of “T” connected to the chestpiece 10, the first headset unit 20, and the second headset unit 30. At this time, the other end of each of the first connecting tube 41 and the second connecting tube 42 is connected to the chestpiece 13 through a connecting body C at the same time.

As the first connecting tube 41 and the second connecting tube 42 of the connection unit 40 are connected to the chestpiece 13, the first headset tube 23, and the second headset tube 33, the auscultation sound auscultated from the chestpiece 10 is separated to the first headset unit 20 and the second headset unit 30 at the same time and delivered thereto.

According to another exemplary embodiment of the inventive concept, auscultating operation of the stethoscope 1 according to an exemplary embodiment will be described referring to FIG. 1.

A doctor contacts any one of the diaphragm 11 or the bell 12 of the chestpiece 10 to the body while wearing the first earpieces 21 on the ears. The auscultation sound auscultated through the chestpiece 10 is delivered to the first headset tube 23 and the second headset tube 33 connected to the first connecting tube 41 and the second connecting tube through the auscultating tube 13, the connecting body C, and the first and the second connecting tubes 41, 42 in order.

The auscultation sound guided to the first headset tube 23 and the second headset tube 33 is delivered to the doctor's ears and the patient's ears through the first binaurals 22 and the second binaurals 32 and the first earpieces 23 and the second earpieces 33 in order. As such, the patient as well as the doctor may listen to the auscultation sound, and the patient may have increased reliability on the examine result.

Meanwhile, the connection unit 40 has a shape of “T” among the chestpiece 10, the first headset unit 20, and the second headset unit 30, but the embodiment is not restricted thereto. For example, the chestpiece 10 and the first headset unit 20 may be connected with each other through the auscultation tube 13 and the first headset tube 23, and the connection unit 40 may connect the second headset unit 30 and the chestpiece 10 with each other in wireless by a Bluetooth. In this case, it is possible for a patient to listen to the auscultation sound only when he wishes to listen by wearing the second headset unit 30 with the Bluetooth function on. That is, the Bluetooth included in the connection unit 40 may have on/off system.

While the present invention has been particularly shown and described with reference to exemplary embodiments thereof, it will be understood that various changes in form and details may be made therein without departing from the spirit and scope of the following claims.

### INDUSTRIAL USABILITY

The present invention is applicable to a stethoscope.

What is claimed is:

1. A stethoscope comprising:
a chestpiece for auscultating a patient;
a first headset unit enabling a doctor to listen to auscultation sound auscultated by the chestpiece;
a second headset unit enabling a patient to listen to the auscultation sound; and
a connection unit for connecting the chestpiece, the first headset unit, and the second headset unit with each other and guiding the auscultation sound to the first headset unit and the second headset unit.

2. The stethoscope of claim 1, wherein the connection unit is prepared among the chestpiece, the first headset unit, and the second headset unit and includes a first connection unit and a second connection unit connected to the chestpiece by each one terminal and to the first headset unit and the second headset unit by each other terminal.

3. The stethoscope of claim 1, wherein the connection unit has a shape of hallow hole “T” each end being connected to the chestpiece, the first headset unit, and the second headset unit and divides the auscultation sound from the chestpiece to deliver the same to the first headset unit and the second headset unit at the same time.

4. The stethoscope of claim 1, wherein the connection unit includes a Bluetooth connecting the second headset unit and the chestpiece in wireless.

5. A stethoscope comprising:
a chestpiece for auscultating a patient;
a first head set unit and a second headset unit for delivering the auscultation sound from the chestpiece; and
a connection unit prepared among the chestpiece, the first headset unit, and the second headset unit and guiding the auscultation sound to each of the first headset unit and the second headset unit by separating the auscultation sound.

6. The stethoscope of claim 5, wherein the connection unit includes a first connecting tube and a second connecting tube which are separated such that one end of each is connected to the chestpiece and the other end of each is connected to the first headset unit and the second headset unit, respectively, and the each one end of the first connecting tube and the second connecting tube is connected to the chestpiece across a connecting body.

7. A stethoscope comprising:
a chestpiece for auscultating a patient;
a first headset unit enabling a doctor to listen to auscultation sound auscultated by the chestpiece;
a second headset unit enabling a patient to listen to the auscultation sound; and
a connection unit for connecting the chestpiece and the second headset unit with each other and guiding the auscultation sound to the second headset unit.

8. The stethoscope of claim 7, wherein the connection unit is a shape of hollow hole “T” connected to the chestpiece, the first headset unit, and the second headset unit, which enables to separate the auscultation sound from the chestpiece to deliver the sound to the first headset unit and the second headset unit at the same time.

9. The stethoscope of claim 7, wherein the connection unit includes a Bluetooth connecting the second headset unit and the chestpiece in wireless.
